(19) United States
(12) Patent Application Publication
(10) Pub. No.: US 2025/0164429 A1
(43) Pub. Date: May 22, 2025

(54) SYSTEMS AND METHODS FOR AN ATOMIC-THIN TWO-DIMENSIONAL MATERIAL BASED PORTABLE GAS SENSOR DEVICE

(71) Applicant: University of Maryland, College Park, College Park, MD (US)

(72) Inventors: Cheng Gong, Potomac, MD (US); Ti Xie, Greenbelt, MD (US); John Robertson Rzasa, University Park, MD (US)

(21) Appl. No.: 18/895,822

(22) Filed: Sep. 25, 2024

Related U.S. Application Data
(60) Provisional application No. 63/584,943, filed on Sep. 25, 2023.

(51) Int. Cl. G01N 27/12; G01N 33/12

(52) U.S. Cl. CPC G01N 27/122; G01N 33/12

(57) ABSTRACT

A system for determining food freshness includes a gas sensor including an atomic-thin two-dimensional material; an instrumentation circuit configured to supply a constant current to the atomic-thin two-dimensional material; an analog-to-digital converter configured to convert a voltage to a digital signal representative of the voltage; a transceiver configured to transmit the digital signal to a remote device; a processor; and a memory. The memory includes instructions stored thereon, which, when executed by the processor, cause the system to: apply a constant current across the atomic-thin two-dimensional material; determine a compliance voltage across the atomic-thin two-dimensional material; sense a change in the compliance voltage based on exposing the atomic-thin two-dimensional material to a gas; transmit the sensed change to the remote device; determine if the sensed change represents a first value greater than a threshold value; and determine an amount of the gas based on the determination.

### CROSS-REFERENCE TO RELATED APPLICATION AND CLAIM OF PRIORITY

This application claims the benefit of, and priority to, U.S. Provisional Patent Application No. 63/584,943, filed on Sep. 25, 2023, the entire contents of which are hereby incorporated herein by reference.

### GOVERNMENT SUPPORT

This invention was made with government support under 49100423C0022, 49100423C0011, and 49100424C0007 awarded by the National Science Foundation. The government has certain rights in the invention.

### TECHNICAL FIELD

The present disclosure relates generally to the field of food freshness. More specifically, the present disclosure provides systems and methods for detecting food freshness using a portable atomic-thin two-dimensional material-based sensor.

### BACKGROUND

In 2010, it was estimated that food loss and waste were estimated to be about 31 percent of the total food supply in the U.S. This is equivalent to 133 billion pounds and $162 billion in waste or loss. The top three groups in terms of share of the total value of food loss are meat (30 percent, including poultry and fish), vegetables (19 percent), and dairy products (17 percent). Per the USDA, 21.7 percent of meat that enters the retail market has been lost as “not eaten” at the consumer level and 4.6 percent at the retailer level.

The common method for companies to suggest the edibility of meat products is via the “sell by” tags on product packages, which is often inaccurate because it does not fully consider the history of a specific product on sale. For example, freshness depends on a variety of factors including, but not limited to, the amount of time a meat product stayed in transportation, the temperature of the transportation environment, etc. In addition, other factors after the product is purchased by a consumer can affect the freshness including, but not limited to, refrigerator temperature, the treatment that leftover meat is subjected to, etc. Without such detailed information, “sell by” tags can convey very limited information to consumers. Based solely on “sell by” tags, spoiled meat may be eaten, or good meat may be discarded.

People can generally smell unpleasant odors when meat becomes spoiled. Thus, gas detection is a practical way for food freshness monitoring. Spoiled food products, such as meats, usually emit or release ammonia (NH₃), trimethylamine (N(CH₃)₃), and/or hydrogen sulfide (H₂S). Although gas sensors exist in industry for other applications (e.g., for toxic gas detection in nanofabrication clean rooms), these facilities/equipment are relatively bulky, of low sensitivity, and/or expensive, thereby making them impractical for food freshness tests in the consumer domain.

While there are a handful of meat freshness sensors on the market for consumers they are relatively expensive and easily malfunction when getting close to the spoiled meats due to the strong chemical reactions between gases and the sensor materials, which are typically metal-oxide nanoparticles.

Accordingly, there is a need for portable and inexpensive systems and devices that do not rely on chemical reactions and can help a user quickly determine the freshness of food products. Such systems and devices will aid in food safety and minimize food waste.

Accordingly, there is interest in enabling the determination of food freshness.

### SUMMARY

An aspect of the present disclosure provides a system for determining food freshness, which includes a gas sensor including an atomic-thin two-dimensional material; an instrumentation circuit configured to supply a constant current to the atomic-thin two-dimensional material; an analog-to-digital converter (ADC) configured to convert a voltage to a digital signal representative of the voltage; a transmitter configured to transmit the digital signal to a remote device; a processor; and a memory. The memory includes instructions stored thereon, which, when executed by the processor, cause the system to: apply a constant current across the atomic-thin two-dimensional material, wherein the atomic-thin two-dimensional material has a resistance; determine a compliance voltage across the atomic-thin two-dimensional material; sense a change in the compliance voltage based on exposing the atomic-thin two-dimensional material to a gas; convert the change in the compliance voltage to a digital signal using the ADC; transmit, by the transmitter, the digital signal to the remote device; determine if the digital signal represents a first value greater than a threshold value; determine an amount of the gas the determination that the digital signal represents a first value greater than a threshold value; and cause the remote device to display the amount of the gas on a display of the remote device.

In accordance with aspects of the disclosure, the system may further include a second gas sensor. The instructions, when executed by the processor, may further cause the system to: detect a second gas using the second gas sensor.

In accordance with aspects of the disclosure, the display of the amount of gas may be in real-time.

In accordance with aspects of the disclosure, the gas may be from a food sample.

In accordance with aspects of the disclosure, the instructions, when executed by the processor, may further cause the system to determine an amount of freshness of the food sample based on the determined amount of gas.

In accordance with aspects of the disclosure, the instrumentation circuit may include a galvanostat circuit.

In accordance with aspects of the disclosure, the instrumentation circuit may include a transimpedance amplifier configured to convert the current into a proportional voltage.

In accordance with aspects of the disclosure, the system may further include a battery and a charge controller.

In accordance with aspects of the disclosure, the instructions, when executed by the processor, may further cause the system to: calibrate the system based on an ambient temperature and a steady-state ambient gas mixture in the absence of the food sample.

In accordance with aspects of the disclosure, the transmitter may transmit the signal wirelessly to the remote device.

An aspect of the present disclosure provides a processor-implemented method for determining food freshness. The method may include: applying a constant current across a graphene element of a portable sensor; determining a compliance voltage across the atomic-thin two-dimensional material; sensing a change in the compliance voltage based on exposing the atomic-thin two-dimensional material to a gas; converting the change in the compliance voltage to a digital signal using an analog-to-digital (ADC) converter of the portable sensor; transmitting, by the transmitter, the digital signal to a remote device; determine if the digital signal represents a first value greater than a threshold value; determining an amount of the gas based on the determination that the digital signal represents a first value greater than a threshold value; and displaying the amount of the gas on a display of a remote device. The atomic-thin two-dimensional material has a resistance.

In accordance with aspects of the disclosure, the method may further include determining an amount of freshness of the food sample based on the determined amount of gas.

In accordance with aspects of the disclosure, the method may further include causing the remote device to display in real-time the determined amount of freshness of the food sample.

In accordance with aspects of the disclosure, the method may further include calibrating the portable sensor based on an ambient temperature and a steady-state ambient gas mixture in the absence of the food sample.

In accordance with aspects of the disclosure, the transmitter may transmit the signal wirelessly to the remote device.

In accordance with aspects of the disclosure, the determined freshness may be further based on a percentage change of resistance in the atomic-thin two-dimensional material over a measurement period.

In accordance with aspects of the disclosure, the method may further include maintaining the current applied to the atomic-thin two-dimensional material at about 5 μA.

In accordance with aspects of the disclosure, the method may further include converting by a transimpedance amplifier the current into a proportional voltage.

In accordance with aspects of the disclosure, the method may further include applying the current to the atomic-thin two-dimensional material by a three electrode potentiostat circuit, where a counter electrode and a reference electrode are tied together.

An aspect of the present disclosure provides a system for determining food freshness, including a plurality of portable gas sensors configured to sense a gas mixture. Each sensor of the plurality of portable gas sensors includes: a graphene element configured to provide a sensed amount of a gas when exposed to a food sample; a transmitter configured to communicate the sensed amount of gas; and a display configured to display a freshness of the food sample. Each sensor of the plurality of portable gas sensors is located at a different geographical location, such as in different locations of a supply chain used for transporting food. The system further includes a processor; and a memory, including instructions stored thereon, which, when executed by the processor, cause the system to: receive from at least one sensor of the plurality of portable gas sensors a sensed amount of gas; determine a food freshness based on the sensed amount of gas; cause the at least one sensor of the plurality of portable gas sensors to display the determined food freshness; and in a case where the food freshness is below a threshold value, provide an indication of which sensor of the plurality of portable gas sensors has the food freshness below the threshold value.

Further details and aspects of exemplary embodiments of the present disclosure are described in more detail below with reference to the appended figures.

### BRIEF DESCRIPTION OF THE DRAWINGS

A better understanding of the features and advantages of the present disclosure will be obtained by reference to the following detailed description that sets forth illustrative embodiments, in which the principles of the present disclosure are utilized, and the accompanying drawings of which:

FIG. 1 is a diagram of an exemplary device for determining food freshness, in accordance with examples of the present disclosure;

FIG. 2 is a block diagram of a controller for the system of FIG. 1, in accordance with aspects of the present disclosure;

FIG. 3 is a diagram of a gas sensor of the system of FIG. 1, in accordance with aspects of the present disclosure;

FIG. 4 is an illustration of a portable sensor device of the system of FIG. 1, in accordance with aspects of the present disclosure;

FIG. 5 is a diagram of a printed circuit board of the portable sensor device of FIG. 4, in accordance with aspects of the present disclosure;

FIG. 6 is an illustration of an example embodiment of a handheld device for determining food freshness, in accordance with aspects of the present disclosure;

FIG. 7 is a diagram of a system for determining food freshness in two or more locations, in accordance with aspects of the present disclosure; and

FIG. 8 is a flow diagram of a method for determining food freshness using the system of FIG. 1, in accordance with aspects of the present disclosure.

### DETAILED DESCRIPTION

The present disclosure relates generally to the field of food freshness. More specifically, the present disclosure provides systems and methods for detecting food freshness.

Although the present disclosure will be described in terms of specific examples, it will be readily apparent to those skilled in this art that various modifications, rearrangements, and substitutions may be made without departing from the spirit of the present disclosure. The scope of the present disclosure is defined by the claims appended hereto.

For the purpose of promoting an understanding of the principles of the present disclosure, reference will now be made to exemplary embodiments illustrated in the drawings, and specific language will be used to describe the same. It will nevertheless be understood that no limitation of the scope of the present disclosure is thereby intended. Any alterations and further modifications of the novel features illustrated herein, and any additional applications of the principles of the present disclosure as illustrated herein, which would occur to one skilled in the relevant art and having possession of this disclosure, are to be considered within the scope of the present disclosure.

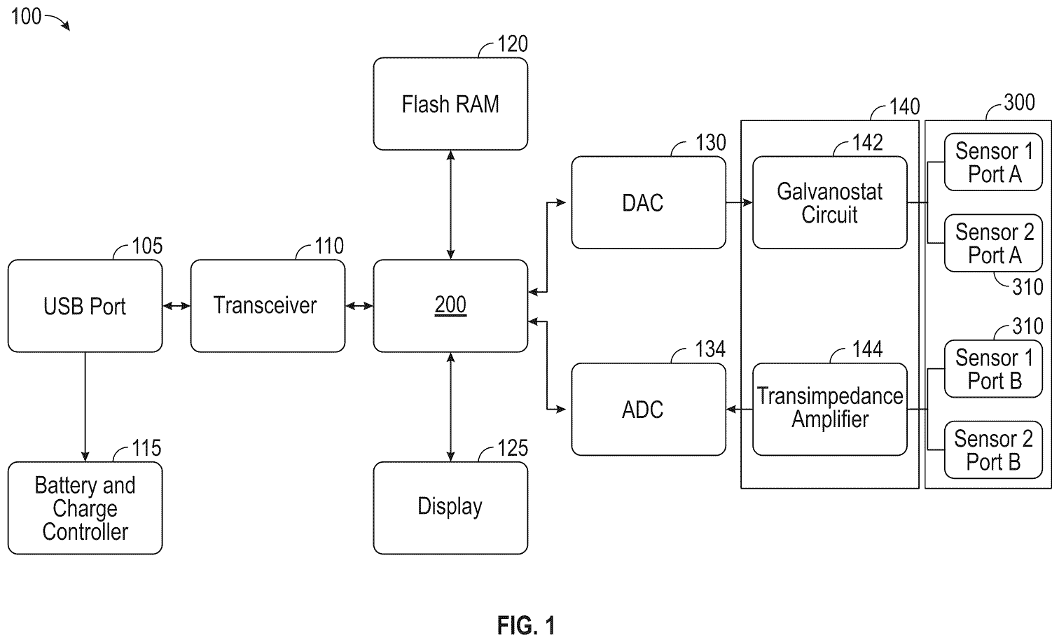

Referring to FIG. 1, a diagram of an example device 100 for determining food freshness is shown.

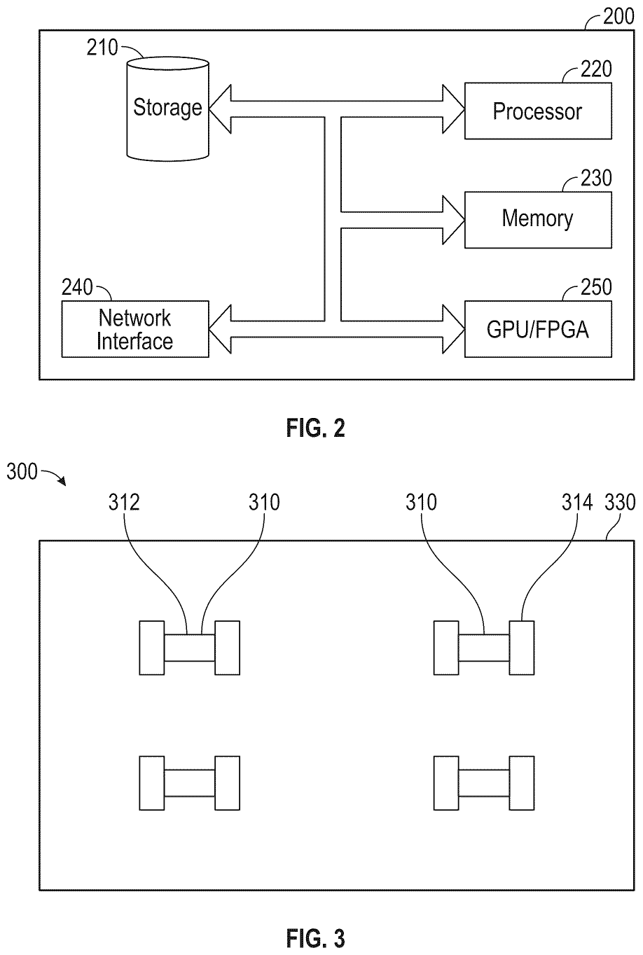

FIG. 1 illustrates a system 100 for determining food freshness. The system 100 generally includes an atomic-thin two-dimensional (2D) material-based gas sensor 300 (FIG. 3) configured to generate a signal when subjected to a gas, an instrumentation circuit 140 (FIG. 5) configured to supply a constant current to the sensor 300, an analog-to-digital converter (ADC) 134 configured to convert a voltage to a digital signal representative of the voltage, a transmitter 110 configured to transmit the digital signal to a remote device, and a controller 200 (FIG. 2). The transceiver 110 may include, for example, a USB serial converter and/or a wireless transceiver. In aspects, system 100 may further include a USB port 105 (FIG. 5) configured for communicating with another device such as a PC or a mobile device and/or providing a charge to a battery. The ADC 134 may be 16, 18, 24, or 32-bit. In aspects, system 100 may include flash RAM 120 configured for storing, for example, operating instructions, calibrations, and/or data captured during the testing of a food sample.

The instrumentation circuit 140 generally includes a galvanostat circuit 142 and a transimpedance amplifier 144. The transimpedance amplifier 144 is configured to convert the current into a proportional voltage. In aspects, to measure the change in resistance across the gas sensor 300, a constant current is applied by a three-electrode potentiostat circuit where the counter and reference electrodes are tied together. The potentiostat circuit acts as a control unit that adjusts the potential of the working electrode relative to the reference electrode. It generally ensures that the voltage applied to the working electrode is maintained at a set value. The potentiostat circuit also measures the current flowing between the working electrode and the counter electrode.

When the resistance changes, a compliance voltage in the galvanostat circuit 142 changes in order to keep the current measured by the transimpedance amplifier 144 the same. This change in voltage is used to measure the level of gas incident on the sensor 300. The galvanostat circuit 142 may include a high-resolution digital-to-analog converter (e.g., DAC 130) for generating the voltage across the sensor 300 and the current flowing through the sensor 300. The DAC 130 may be 16, 18, 24, or 32-bit.

A control loop in the controller 200 (e.g., a field-programmable gate array) adjusts the output voltage of DAC 130 to maintain whatever the desired current set point is. Due to the small size of the sensor 300, the smallest possible current must be used in order to prevent ohmic heating of the sensor, which will cause an unwanted change in resistance. Therefore, the sensitivity of the galvanostat circuit 142 should be very high. The system 100 can reliably maintain a set point of about 5 μA, but tests have shown that levels on the order of 200 μA are sufficient. The galvanostat circuit 142 may also include two or more independent channels in case sample/control tests are conducted, or multiple gases are simultaneously measured. In aspects, multiple channels may be used to detect a multitude of gases simultaneously.

In aspects, the system 100 may further include a display 125, such as a touch screen, an LCD or other suitable display. It is contemplated that the display 125 may be remote, such as a display on a mobile device or a computer. The display may be configured to display the amount of the gas sensed by the sensor 300. The display may display the results in real time. In aspects, the display may be configured to display an amount of freshness of the food sample based on the determined amount of gas.

In aspects, the system may further include a battery and a charge controller 115 configured to provide portable power for the system 100.

Referring now to FIG. 2, exemplary components in the controller 200 in accordance with aspects of the present disclosure include, for example, a database 210, one or more processors 220, at least one memory 230, and a network interface 240. In aspects, the controller 200 may include a graphical processing unit (GPU) 250, which may be used for processing machine learning models.

Database 210 can be located in storage. The term “storage” may refer to any device or material from which information may be capable of being accessed, reproduced, and/or held in an electromagnetic or optical form for access by a computer processor. Storage may be, for example, volatile memory such as RAM, non-volatile memory, which permanently holds digital data until purposely erased, such as flash memory, magnetic devices such as hard disk drives, and optical media such as a CD, DVD, Blu-ray disc, or the like. In various embodiments, data may be stored on the controller 200, including, for example, user preferences, historical data, and/or other data. The data can be stored in database 210 and sent via the system bus to the processor 220.

As will be described in more detail later herein, the processor 220 executes various processes based on instructions that can be stored in the server memory 230 and utilizing the data from the database 210. The illustration of FIG. 2 is exemplary, and it will be understood by persons skilled in the art that other components may exist in a controller 200. Such other components are not illustrated in FIG. 2 for clarity of illustration.

FIG. 3 is a diagram of a gas sensor 300. Gas sensor 300 can quickly detect gaseous molecules emitted from spoiled food products, e.g., meats, without requiring chemical reactions. Gas sensor 300 provides the technical benefit of being sensitive, inexpensive, and robust. The gas sensor 300 may include one or more sensor elements 310 that each include atomic-thin 2D material 312 disposed on a substrate 330, and a pair of conductive terminals 314 on each end portion of the atomic-thin 2D material 312. The atomic-thin 2D materials may include, for example, graphene, Molybdenum disulfide (MoS₂), tungsten disulfide (WS₂), and/or Tungsten diselenide (WSe₂). Gas sensor 300 may be configured to detect gas mixtures comprising, for example, ammonia (NH₃), hydrogen sulfide (H₂S), trimethylamine (N(CH₃)₃), and/or sulfur dioxide (SO₂). Gas sensor 300 may include a plurality of sensor elements 310, each configured to detect a different gas in a gas mixture.

Each sensor of the one or more sensor elements 310 changes its resistance as it is exposed to a particular gas. As the gas concentration decreases, the resistance will revert to its original state, so the sensor should be reusable.

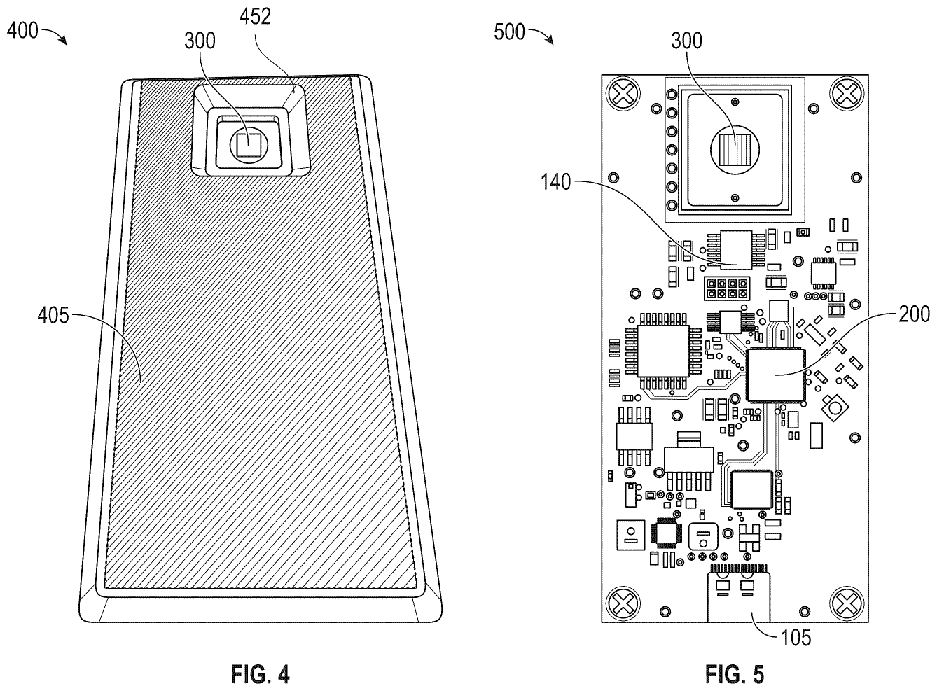

Referring to FIGS. 4 and 5, a portable sensor device 400 of system 100 is shown. Portable sensor device 400 is configured to work in conjunction with a user device such as a smartphone. Device 400 includes a housing 405. Disposed within housing 405 is circuit board 500. Device 400 generally includes many of the same components as system 100, except for the display 125. The device 400 includes an opening 452 where the gas sensor 300 is accessible to the air. The opening 452 enables the user to touch food against the device 400 to bring it close to the sensor 300 without contaminating the sensor 300 with the food.

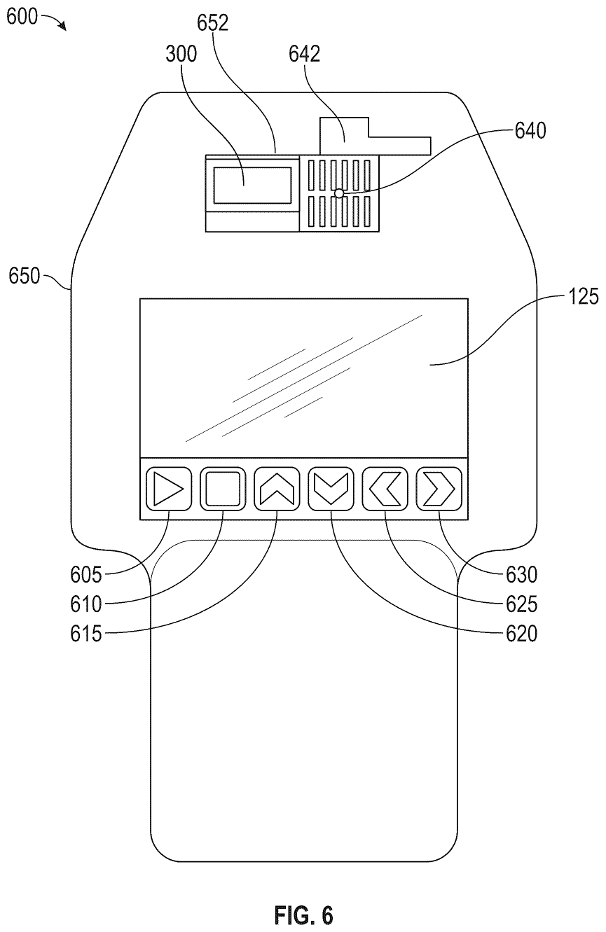

FIG. 6 is a handheld device 600 for determining food freshness. The handheld device 600 includes many of the same components as system 100. The device 600 includes a housing 650 having an opening 652 where the gas sensor 300 is accessible to the air. The opening 652 enables the user to touch food against the device 600 to bring it close to the sensor 300 without contaminating the sensor 300 with the food. Device 600 further includes a display 125 configured for displaying the determined food freshness and controls 605, 610, 615, 620, 625, 630 configured for navigating a user interface. For example, controls 605, 610, 615, 620, 625, 630 may be used to change a threshold for determining the food freshness, or may be used to allow a user to store or load data. Device may include a low insertion force socket 640 configured for holding sensor 300. The low insertion force socket 640 may include a handle 642 configured for gripping or releasing the sensor 300.

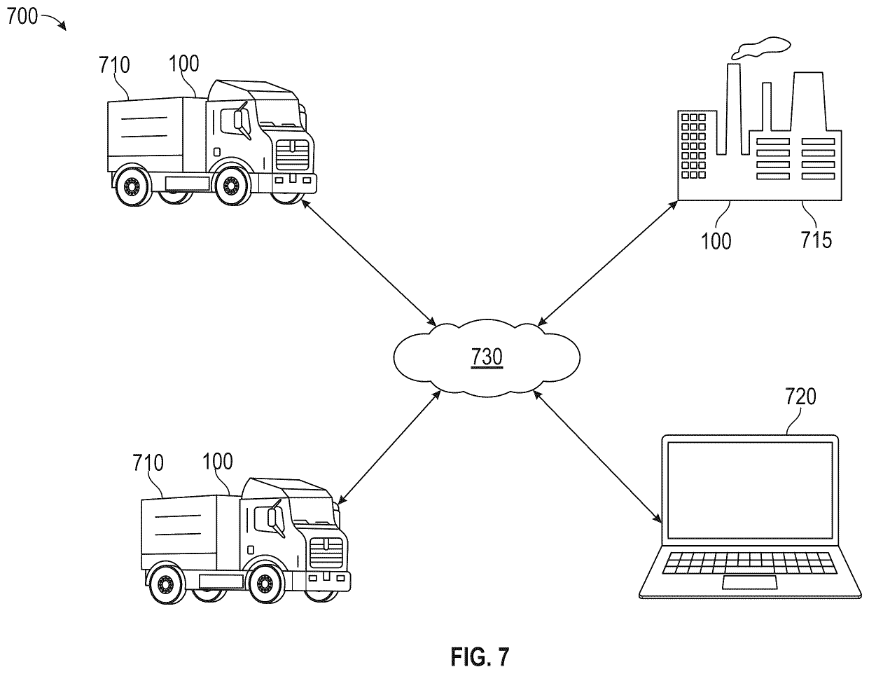

FIG. 7 is a diagram of an exemplary system 700 for determining food freshness, such as at different locations along a supply chain used for the transportation of food. System 700 generally includes a plurality of portable gas sensor systems 100 configured to sense a gas mixture. Each sensor system of the plurality of portable gas sensor systems 100 may be located at a different geographical location. For example, each of the sensor systems 100 may be located in a food truck 710 (e.g., a food delivery truck or a food service truck) or at a location such as a food processing plant 715 (or, for example a distribution center, and supermarket). Each sensor system 100 may communicate via network 730. System 700 may include a user device 720, such as a computer or a mobile device configured for receiving data from each sensor system of the plurality of portable gas sensor systems 100. In aspects, system 700 may receive a sensed amount of gas from at least one sensor of the plurality of portable gas sensors. In aspects, system 700 may determine a food freshness based on the sensed amount of gas. In aspects, system 700 may cause the at least one sensor of the plurality of portable gas sensors to display the determined food freshness. In aspects, system 700 may, in a case where the food freshness is below a threshold value, provide an indication of which of the plurality of portable gas sensors has the food freshness below the threshold value to identify the food along the supply chain that is below the acceptable grade or is spoiled.

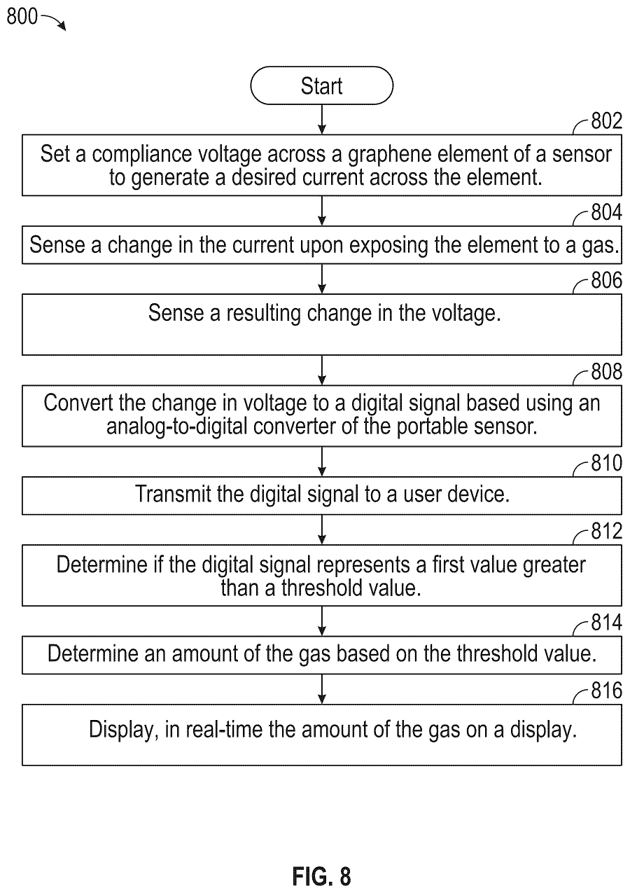

Referring to FIG. 8, a flow diagram for a method 800 for determining food freshness is shown. Although the blocks of FIG. 8 are shown in a particular order, the blocks need not all be performed in the illustrated order, and certain blocks can be performed in another order. For example, FIG. 8 will be described below, with a controller 200 of FIG. 2 performing the operations. In aspects, the operations of FIG. 8 may be performed all or in part by another device, for example, a server, a user device, and/or a computer system. These variations are contemplated to be within the scope of the present disclosure.

Initially, at block 802, the controller 200 causes the system 100 to set a compliance voltage across sensor element 310 (FIG. 3) of system 100. In aspects, the compliance voltage may be set using the DAC 130 (FIG. 1) to generate a current across sensor element 310.

In aspects, prior to use, the system 100 may be calibrated based on an ambient temperature and a steady-state ambient gas mixture in the absence of the food sample.

At block 804, the controller 200 causes the system 100 to sense a change in the current based on exposing the sensor element 310 to a gas by converting the current to a proportional voltage. The current may be changed to the proportional voltage using a transimpedance amplifier 144 and the ADC 134 of system 100.

At block 806, the controller 200 causes the system 100 to sense a change in the resulting compliance voltage based on exposing the sensor element 310 to the gas. In aspects, the resulting change in compliance voltage may be sensed using the DAC 130 in order to maintain a constant current, and then reporting this change to the controller 200. The constant current, for example, may be about 5 μA.

In aspects, the controller 200 may cause the system 100 to determine a first voltage (the voltage prior to the sensor element 310 being exposed to a gas) across the sensor element 310. For example, when no gas is present, the sensor element 310 has a voltage across it that is proportional to the resistance of the sensor element 310 and the constant current. The system 100 may determine this voltage using the transimpedance amplifier 144 to convert the current into a proportional voltage. For example, a food sample may be brought near the sensor element 310 of sensor 300 to expose the sensor element 310 to a gas and/or gas mixture emanating from the food sample. When exposed to the gas and/or gas mixture, the resistance of the sensor element 310 will change causing the voltage across the sensor element 310 to change (the current is constant). At block 808, the controller 200 causes the system 100 to convert the change in voltage to a digital signal based using an analog-to-digital converter (ADC) 134. At block 810, the controller 200 causes the system 100 to transmit, by the transceiver, the digital signal to the remote device. For example, the transceiver may be a USB transceiver, or a wireless transceiver such as Bluetooth® or WIFI®.

At block 812, the controller 200 causes the system 100 to determine if the digital signal represents a first value greater than a threshold value. For example, the digital signal may represent a time series of values. The values are compared to a threshold and if the values exceed the threshold, then the system 100 would determine that the digital signal represents a first value greater than a threshold value.

At block 814, the controller 200 causes the system 100 to determine an amount of the gas based on the determination that the digital signal represents a first value greater than a threshold value. In aspects, the controller 200 may cause the system 100 to determine an amount of freshness of the food sample based on the determined amount of gas. The controller 200 may cause the system 100 to display an indication of the food freshness.

At block 816, the controller 200 causes the system 100 to cause the remote device to display the amount of the gas on a display. For example, the amount of gas may be displayed on a remote device or on a display of the system 100. For example, the concentration of gases in rotten meat varies depending on the stage of decomposition and environmental conditions. Common gases include hydrogen sulfide (H₂S), typically found in parts per million (ppm) concentrations around 10-50 ppm in early stages, but potentially much higher as decomposition progresses. Ammonia (NH₃) is also present in ppm levels, generally ranging from 10-100 ppm or more. Methane (CH₄) is found in smaller amounts, usually a few ppm to a few hundred ppm. Carbon dioxide (CO₂) can be present in higher concentrations, reaching several thousand ppm due to microbial activity. The exact levels of these gases can differ significantly based on factors such as meat type, temperature, moisture, and microbial activity. In aspects, system 100 can be adjusted to account for varying requirements for food freshness.

Certain embodiments of the present disclosure may include some, all, or none of the above advantages and/or one or more other advantages readily apparent to those skilled in the art from the drawings, descriptions, and claims included herein. Moreover, while specific advantages have been enumerated above, the various embodiments of the present disclosure may include all, some, or none of the enumerated advantages and/or other advantages not specifically enumerated above.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in various embodiments,” “in some embodiments,” or “in other embodiments” may each refer to one or more of the same or different example embodiments provided in the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed is:

1. A system for determining food freshness, the system comprising:
a gas sensor comprising an atomic-thin two-dimensional material;
an instrumentation circuit configured to supply a constant current to the atomic-thin two-dimensional material;
an analog-to-digital converter (ADC) configured to convert a voltage to a digital signal representative of the voltage;
a transceiver configured to transmit the digital signal to a remote device;
a processor; and
a memory, including instructions stored thereon, which, when executed by the processor, cause the system to:
apply a constant current across the atomic-thin two-dimensional material, wherein the atomic-thin two-dimensional material has a resistance;
determine a compliance voltage across the atomic-thin two-dimensional material;
sense a change in the compliance voltage based on exposing the atomic-thin two-dimensional material to a gas;
convert the change in compliance voltage to a digital signal using the ADC;
transmit, by the transceiver, the digital signal to the remote device;
determine that the digital signal represents a first value greater than a threshold value;
determine an amount of the gas based on the determination that the digital signal represents a first value greater than a threshold value; and
cause the remote device to display the amount of the gas on a display of the remote device.

2. The system of claim 1, wherein the system further includes a second gas sensor, and wherein the instructions, when executed by the processor, further cause the system to detect a second gas using the second gas sensor.

3. The system of claim 1, wherein the displaying of the amount of gas is in real-time.

4. The system of claim 1, wherein the gas is from a food sample.

5. The system of claim 1, wherein the instructions, when executed by the processor, further cause the system to determine an amount of freshness of the food sample based on the determined amount of gas.

6. The system of claim 1, wherein the instrumentation circuit includes a galvanostat circuit.

7. The system of claim 1, wherein the instrumentation circuit includes a transimpedance amplifier configured to convert the current into a proportional voltage.

8. The system of claim 1, wherein the system further includes a battery and a charge controller.

9. The system of claim 1, wherein the instructions, when executed by the processor, further cause the system to:
calibrate the system based on an ambient temperature and a steady-state ambient gas mixture in the absence of the food sample.

10. The system of claim 1, wherein the transceiver transmits the signal wirelessly to the remote device.

11. A processor-implemented method for determining food freshness, the method comprising:
applying a constant current across an atomic-thin two-dimensional material of a portable sensor, wherein the atomic-thin two-dimensional material has a resistance;
determining a compliance voltage across the atomic-thin two-dimensional material;
sensing a change in compliance voltage based on exposing the atomic-thin two-dimensional material to a gas;
converting the change in compliance voltage to a digital signal using an analog-to-digital (ADC) converter of the portable sensor;
transmitting, by the transceiver, the digital signal to a remote device;
determine if the digital signal represents a first value greater than a threshold value;
determining an amount of the gas based on the determination that the digital signal represents a first value greater than a threshold value; and
displaying the amount of the gas on a display of a remote device.

12. The processor-implemented method of claim 11, further comprising:
determining an amount of freshness of the food sample based on the determined amount of gas.

13. The processor-implemented method of claim 11, further comprising:
causing the remote device to display in real-time the determined amount of freshness of the food sample.

14. The processor-implemented method of claim 11, further comprising:
calibrating the portable sensor based on an ambient temperature and a steady-state ambient gas mixture in the absence of the food sample.

15. The processor-implemented method of claim 11, wherein the transceiver transmits the signal wirelessly to the remote device.

16. The processor-implemented method of claim 11, wherein the determined freshness is further based on a percentage change of resistance in the atomic-thin two-dimensional material over a measurement period.

17. The processor-implemented method of claim 11, further comprising:
maintaining the current applied to the atomic-thin two-dimensional material at about 5 μA.

18. The processor-implemented method of claim 11, further comprising:
converting by a transimpedance amplifier the current into a proportional voltage.

19. The processor-implemented method of claim 11, further comprising:
applying the current to the atomic-thin two-dimensional material by a three electrode potentiostat circuit, where a counter electrode and a reference electrode are tied together.

20. A system for determining food freshness, the system comprising:
a plurality of portable gas sensors configured to sense a gas mixture, wherein each sensor of the plurality of portable gas sensors is located in a different geographical location and includes:
an atomic-thin two-dimensional material configured to provide a sensed amount of a gas when exposed to a food sample;
a transceiver configured to communicate the sensed amount of gas; and
a display configured to display a freshness of the food sample, wherein each of the plurality of portable gas sensors are located at a different geographical location;
a processor; and
a memory, including instructions stored thereon, which, when executed by the processor, cause the system to:
receive from at least one sensor of the plurality of portable gas sensors a sensed amount of gas;
determine a food freshness based on the sensed amount of gas;
cause the at least one sensor of the plurality of portable gas sensors to display the determined food freshness; and
in a case where the food freshness is below a threshold value, provide an indication of which sensor of the plurality of portable gas sensors has the food freshness below the threshold value.
